(19) United States
(12) Patent Application Publication
(10) Pub. No.: US 2024/0239795 A1
(43) Pub. Date: Jul. 18, 2024

(54) NOVEL AZAINDOLE DERIVATIVES AS ANTIVIRAL AGENTS

(71) Applicants: Centre National de la Recherche Scientifique, PARIS (FR); Université de Montpellier, MONTPELLIER (FR)

(72) Inventors: Laurence BRIANT, GALLARGUES-LE-MONTUEUX (FR); Eric BERNARD; Camille CLOP, FOURNES (FR)

(21) Appl. No.: 18/563,284

(22) PCT Filed: May 23, 2022
(86) PCT No.: PCT/FR2022/050977

§ 371 Date: Nov. 21, 2023

(30) Foreign Application Priority Data
May 21, 2021 (FR) 2105360

(51) Int. Cl. C07D 471/04; A61K 31/437; A61K 31/444; A61K 45/06; A61P 31/14

(52) U.S. Cl. CPC C07D 471/04; A61K 31/437; A61K 31/444; A61K 45/06; A61P 31/14

(57) ABSTRACT

The present invention relates to compounds derived from 7-azaindole useful as inhibitors of AXL kinases for the treatment of viral infections. The present invention also relates to their method of preparation. Specifically, the invention relates to compounds of formula (I): for use in the prevention and/or treatment of viral infections.

### FIELD OF THE INVENTION

The present invention relates to compounds derived from 7-azaindole useful as inhibitors of AXL kinases for the treatment of viral infections. The present invention also relates to their method of preparation.

### TECHNICAL BACKGROUND

AXL is a Receptor Tyrosine Kinase (RTK) belonging to the TAM family composed of TYRO-3, AXL and MER. Initially discovered and demonstrated in patients suffering from chronic myeloid leukemia (CML), the involvement of the AXL receptor in viral infections such as, for example, dengue, the Zika virus, Chikungunya or Ebola fever was described in the literature (Chen, J. et al. Nature Microbiology, 2018, 3, pp.302-309; Fedeli, C. et al. Journal of Virology, 2018, 92:e01613-17; Hastings, A. K. et al., iScience, 2019, 13 pp. 339-350; Meertens, L. et al., Cell Reports, 2017, 18, 3, pp. 324-333). This receptor, by interacting via the Gas6 adapter with phosphatidylserines present in the viral envelopes, promotes the attachment of the enveloped viruses to their cell target. AXL thus stimulates endocytosis of the enveloped viruses in their cell targets. The engagement of AXL during this interaction stimulates the phosphorylation of the AXL intracytoplasmic domain and activates the associated signaling pathways. These signals neutralize the production of interferons and the associated antiviral responses, promoting the escape of these viruses from the immune control of the host.

To date, there are several active kinase inhibitors on AXL (Myer et al., J. Med. Chem. (2015), 59(8): 3593-3608) but none is really selective or specific for this kinase. In a large majority of cases, the activity on AXL is secondary with respect to the desired main activity on MET or MER due to the similarity of sequence between these RTKs. It is in particular the case for bosutinib or cabozantinib, which are multi-targeted kinase inhibitors or MTKIs already on the market, or BMS777607 (currently in clinical phase 2). This is also the case of the 7-azaindole derivative NPS-1034, which has a relatively broad inhibition profile, by inhibiting a large panel of kinases such as AXL/DDR1/FLT3/KIT/MEK/MET/ROS1 and TIE1.

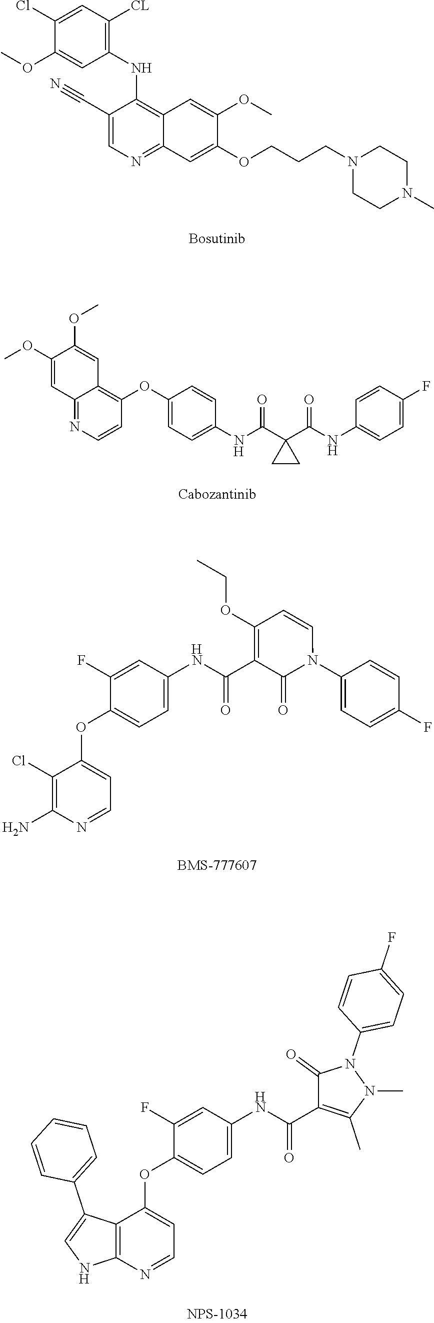

Another AXL kinase inhibitor is R428 (also called BGB324), currently in the clinical phase. This compound—which has a very different structure from kinase inhibitors currently on the market—has also been revealed to be active on other kinases such as ABL/KIT/JAK2-3/LCK/PDGFRB/TIE2.

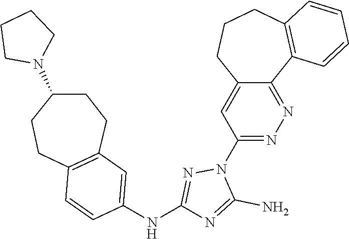

R428/BGB324

There is therefore a need for novel AXL inhibitor compounds which are more selective and effective with respect to viral infections.

The present invention thus proposes novel 7-azaindole derivatives that strongly inhibit AXL kinase for use as antiviral agents.

By virtue of this very specific and unique inhibition profile for this type of structure, these compounds can be used in therapy in the treatment of infections caused by viruses using the AXL receptor to multiply. In order to combat viral infections, by inhibiting the phosphorylation of AXL, these compounds will act both by blocking the endocytosis of the enveloped viruses in their cell target, and by neutralizing the intracellular signals controlled by AXL which inhibit the production of the interferons and of the antiviral responses of the host. The inhibitors of the present invention can thus be used for the treatment of diseases in which AXL are involved, in particular viral infections and in particular viral input in the cells.

### SUMMARY OF THE INVENTION

The present invention thus relates to a compound of formula (I):

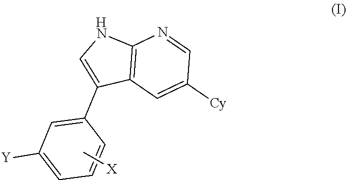

in which
X represents a hydrogen atom or a halogen atom,
Y is selected from —CH₂OH, —CH═NOH, —CH₂NH₂, a heteroaryl aryl optionally mono- or polysubstituted, a heteroaryl optionally mono- or polysubstituted, and -L-(CH₂)p-W,
   L represents —C(O)NH—, —CH₂NH—, —CH═N—, —NHC(O)—, or —CH₂N═CH—,
   p is an integer from 0 to 2,
   when L represents —CH₂NH—, —CH═N—, or —CH₂N═CH—, W is selected from:
   a C₁-C₆ alkyl,
   a C₆-C₁₀ aryl, optionally mono- or polysubstituted; and
   a 5-10 membered heteroaryl containing from 1 to 3 heteroatoms independently selected from N, O and S, said heteroaryl optionally being mono- or polysubstituted;
   when L represents —C(O)NH— or —NHC(O)—, W is selected from:
   a C₆-C₁₀ aryl, optionally mono- or polysubstituted;
   a 5-10 membered heteroaryl containing from 1 to 2 heteroatoms independently selected from N, O and S, said heteroaryl optionally being mono- or polysubstituted; and
   a C₃-C₆ cycloakyl optionally substituted with a C(O)NHR or NHC(O)R group wherein R represents a C₆-C₁₀ aryl optionally substituted with a halogen atom and/or a C₁-C₆ alkyl, in particular the group

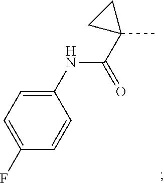

Cy represents a phenyl group or a 5-10 membered heteroaryl group containing from 1 to 3 heteroatoms independently selected from N, O or S, said heteroaryl optionally being substituted with an oxo group, a pharmaceutically acceptable salt thereof or a mixture thereof, for use in the prevention and/or treatment of viral infections.

The present invention also relates to a pharmaceutical composition comprising a compound according to the invention or a pharmaceutically acceptable salt thereof as an active ingredient, and a pharmaceutically acceptable excipient, for use in the prevention and/or treatment of viral infections.

### DETAILED DESCRIPTION

As a general rule, the following terms and definitions are used.

The expression “peptide coupling” in the present invention refers to the reaction for forming an amide —NH—C(O)— bond. The techniques used in this reaction are common to peptide syntheses, that is to say, by activation of a carboxylic acid to react with an amine. The peptide coupling reactions used in the present invention are thus derived from peptide syntheses, and directly applicable to the subject matter of the present invention.

The peptide coupling reactions are well known to a person skilled in the art, and can in particular be carried out using a coupling agent such as, N,N′-dicyclohexylcarbodiimide (DCC) or 1-ethyl-3-(3′-dimethylaminopropyl)carbodiimide hydrochloride (EDC), or N-hydroxy-5-norbornene-2,3-dicarbodiimide), or a benzotriazole (such as O-(1H-benzotriazol-1-yl)-N,N,N′,N′-tetramethyluronium (TBTU), benzotriazol-1-yl-oxytris(dimethylamino)phosphonium hexafluorophosphate (BOP), O-(7-azabenzotriazol-1-yl)-1,2,3-tetramethyluronium hexafluorophosphate (HATU), O-benzotriazol-1-yl-N,N,N′,N′-tetramethyluronium hexafluorophosphate (HBTU), O-benzotriazol-1-yl-tetramethyl tetrafluoroborate (TBTU)), or an N-hydroxybenzotriazole (HOBT)/EDCI mixture, in a solvent such as chloroform, dichloromethane, dichloroethane, ethyl acetate, dimethylformamide (DMF), tetrahydrofuran (THF), dimethylsulfoxide (DMSO), N-methyl pyrrolidinone (NMP), preferably at a temperature of between 20° C. and 150° C.

Alternatively, peptide coupling takes place by first activating the carboxylic acid by transformation into acyl chloride (in particular in the presence of thionyl chloride or acetyl chloride) or a corresponding anhydride (for example in the presence of acetic anhydride or isopropyl), then reaction with the desired amine, preferably in the presence of a base to neutralize the acid released during the reaction (in particular HCl in the case of an acyl chloride).

The term C(O) is equivalent to “C═O”.

The expression “alkyl group” or “alkyl” in the present invention denotes a saturated linear or branched aliphatic group containing 1 to 6 carbon atoms, if not otherwise defined. Examples of alkyl groups covered by the subject matter of the present invention are methyl, ethyl, propyl, butyl, tert-butyl, isopropyl groups.

In some embodiments, it is specified that one or more hydrogen atoms of the alkyl group are optionally replaced by a fluorine atom. In this case, preferably, 1 to 3 hydrogen atoms at most are affected. An example is the group CH₂CF₃.

The expression “aryl group” or “aryl” in the present invention denotes a (mono- or polycyclic) cyclic aromatic group containing between 6 and 10 carbon atoms. Examples of aryl groups covered by the subject matter of the present invention are the phenyl, napthyl, preferably phenyl groups.

The expression “heteroaryl group” or “heteroaryl” in the present invention denotes a 5- to 10-membered (mono- or polycyclic) aromatic cyclic group containing between 2 and 9 carbon atoms and between 1 and 3 heteroatoms independently selected from nitrogen, oxygen or sulfur. Examples of heteroaryl groups are the furan, pyrrole, thiophene, thiazole, isothiazole, imidazole, oxazole, isoxazole, pyrazole, pyridine, pyrazine, pyridazine, pyrimidine, quinoline, indole, quinoxaline, benzofuran, dihydrobenzofuran, benzodioxole, benzotriazole, benzimidazole groups, preferably chosen from pyrrole, imidazole, thiophene, pyridine, pyrimidine, benzofuran, benzodioxole, indole and benzimidazole.

The expression “cycloalkyl” or “cycloalkyl group” denotes a cyclic saturated aliphatic group containing 3 to 6 carbon atoms, if not otherwise defined. Examples of cycloalkyl groups covered by the subject matter of the present invention are cyclopropyl, cyclobutyl, cyclopentyl or cyclohexyl groups. Preferably, it is cyclopropyl.

The expression “halogen atom” in the present invention denotes a fluorine, chlorine, bromine or iodine atom. Preferably, it is bromine or fluorine, in particular fluorine.

The expression “alkoxyl group” or “alkoxyl” in the present invention refers to an oxygen-bonded alkyl group. Examples of alkoxyl groups are methoxy, ethoxy, propoxy, isopropoxy, butoxy, isobutoxy, tert-butoxy groups. Preferably, it is a methoxy or ethoxy group.

The expression “aryloxy group” in the present invention denotes an aryl group bonded to an oxygen atom. Examples of aryloxy groups are phenyloxy groups.

The expression “hydroxyl group” or “hydroxyl” in the present invention refers to: OH.

The expression “oxo group” denotes the substituent: ═O.

The expression “optionally substituted aryl or heteroaryl group” in the present invention refers to an aryl or heteroaryl optionally substituted by one or more (preferably 1 to 4, even more preferably 1 or 2) substituents independently selected from: a halogen atom, a nitro group —(NO₂), a cyano (CN) group, a C₁-C₆ alkoxyl, a C₅-C₁₀ aryloxy, a C₁-C₆ alkyl wherein 1 or more hydrogen atoms is (are) optionally replaced with a fluorine atom, a heteroaryl, a hydroxyl, a C₁-C₆ —CONH-alkyl group, a C₁-C₆ —NHCO alkyl group, and a NR₂R₃ group wherein R₂ and R₃ independently represent a C₁-C₆ alkyl group, or a C₆-C₁₀ aryl group optionally substituted with a halogen atom and/or a C₁-C₆ alkyl.

Preferably, the substituents are independently selected from:
a halogen atom, in particular a fluorine or a chlorine,
an oxo group,
a hydroxyl,
a C₁-C₆ alkoxyl, in particular a methoxy,
a C₁-C₆ alkyl, wherein 1 or several hydrogen atoms is optionally replaced with a fluorine atom, preferably a methyl group or CH₂CF₃, and
a C₆-C₁₀ aryl optionally substituted with a halogen atom (in particular a fluorine or a chlorine) and/or an alkyl C₁-C₆, for example a fluorophenyl (preferably 4-fluorophenyl) or a methylfluorophenyl (for example 4-fluoro-2-methylphenyl);

The expression “7-azaindole” in the present invention refers to 1H-pyrrolo[2,3-b]pyridine:

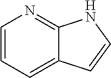

In the present invention, “pharmaceutically acceptable” refers to what is useful in the preparation of a pharmaceutical composition which is generally safe, non-toxic and neither biologically nor otherwise undesirable and which is acceptable for veterinary use as well as human pharmaceutical use.

In the present invention, the expression “pharmaceutical composition” refers to any composition consisting of an effective dose of at least one compound of the invention and at least one pharmaceutically acceptable excipient. Such excipients are selected, depending on the pharmaceutical form and on the desired method of administration, from excipients usually known by the person skilled in the art.

“Pharmaceutically acceptable salts” of a compound refers to salts which are pharmaceutically acceptable, as defined herein, and which have the desired pharmacological activity of the parent compound. Such salts comprise:
(1) hydrates and solvates,
(2) pharmaceutically acceptable acid addition salts formed with pharmaceutically acceptable inorganic acids such as hydrochloric acid, hydrobromic acid, sulfuric acid, nitric acid, phosphoric acid and the like; or formed with pharmaceutically acceptable organic acids such as acetic acid, benzenesulfonic acid, benzoic acid, camphorsulfonic acid, citric acid, ethanesulfonic acid, fumaric acid, glucoheptonic acid, gluconic acid, glutamic acid, glycolic acid, hydroxynaphthoic acid, 2-hydroxyethanesulfonic acid, lactic acid, maleic acid, malic acid, mandelic acid, methanesulfonic acid, muconic acid, 2-naphthalenesulfonic acid, propionic acid, salicylic acid, succinic acid, dibenzoyl-L-tartaric acid, tartaric acid, p-toluenesulfonic acid, trimethylacetic acid, trifluoroacetic acid and the like, or
(3) the pharmaceutically acceptable base addition salts formed when an acid proton present in the parent compound is either replaced with a metal ion, for example an alkali metal ion, an alkaline earth metal ion or an aluminum ion; or coordinated with a pharmaceutically acceptable organic or inorganic base. Acceptable organic bases comprise diethanolamine, ethanolamine, N-methylglucamine, triethanolamine, tromethamine and the like. Acceptable inorganic bases comprise aluminum hydroxide, calcium hydroxide, potassium hydroxide, sodium carbonate and sodium hydroxide.

The expression “enantiomeric mixtures” in the present invention refers to any mixture of enantiomers. The mixture may be racemic, that is to say 50/50 of each enantiomer by weight (w/w), or non-racemic, that is enriched with one or the other of the enantiomers, for example so as to obtain an enantiomeric excess greater than or equal to 95%, preferably greater than or equal to 98%, even more preferably greater than 99%.

The expression “diastereomeric mixtures” in the present invention refers to any mixture of diastereomers regardless of the proportion.

The expression “treatment” applies to all types of animals, preferably to mammals and more preferentially to humans. In the case of the treatment of a non-human animal, the expression refers to a veterinary treatment.

The present invention therefore relates to a compound of formula (I):

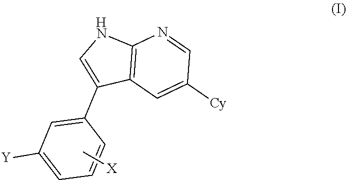

in which
X represents a hydrogen atom or a halogen atom,
Y is selected from —CH₂OH, —CH═NOH, —CH₂NH₂, an aryl optionally mono- or polysubstituted, a heteroaryl optionally mono- or polysubstituted, and -L-(CH₂)p-W,
   L represents —C(O)NH—, —CH₂NH—, —CH═N—, —NHC(O)—, or —CH₂N═CH—,
   p is an integer from 0 to 2,
   when L represents —CH₂NH—, —CH═N—, or —CH₂N—CH—, W is selected from:
   a C₁-C₆ alkyl, preferably a methyl,
   a C₆-C₁₀ aryl, preferably a phenyl, optionally mono- or polysubstituted, advantageously by 1 or 2 groups independently selected from:
   a halogen atom,
   a hydroxyl,
   a C₁-C₆ alkoxyl, preferably a methoxy,
   a C₁-C₆ alkyl, wherein 1 or several hydrogen atoms is (are) optionally replaced with a fluorine atom, preferably a trifluoromethyl, and
   a 5-10 membered heteroaryl comprising from 1 to 3 heteroatoms independently selected from N, O and S, in particular a 5-membered heteroaryl group comprising from 1 to 3 heteroatoms independently selected from N, O and S such as triazole, preferably 1,2,4-triazole;
   a 5-10 membered heteroaryl containing from 1 to 3 heteroatoms independently selected from N, O and S, such as a furan, pyrrole, imidazole, pyrazole, thiophene, pyridine, pyrimidine, benzofuran, benzodioxole, indole, benzimidazole, said heteroaryl optionally being mono- or polysubstituted, advantageously it is optionally substituted with 1 to 4 groups independently selected from:
   a halogen atom,
   an oxo group
   a hydroxyl,
   a C₁-C₆ alkoxyl,
   a C₁-C₆ alkyl, wherein 1 or several hydrogen atoms is (are) optionally replaced with a fluorine atom, and
   a C₆-C₁₀ aryl optionally substituted with a halogen atom and/or a C₁-C₆ alkyl, preferably a fluorophenyl (in particular 4-fluorophenyl) or a terazole substituted with a C₁-C₆ alkyl such as a methyl;
   when L represents —C(O)NH— or —NHC(O)—, W is selected from:
   a C₆-C₁₀ aryl optionally mono- or polysubstituted, advantageously it is optionally substituted with a halogen atom, a hydroxyl, a C₁-C₆ alkyl, or a C₁-C₆ alkoxyl, preferably a fluorine atom, a hydroxyl, a methoxy or a trifluoromethyl, even more preferably a hydroxyl,
   a 5-10 membered heteroaryl comprising from 1 to 2 heteroatoms independently selected from N, O and S, such as a furan, pyrrole, imidazole, pyrazole, thiophene, pyridine, pyrimidine, benzofuran, benzodioxole, indole, benzimidazole, said heteroaryl optionally being mono- or polysubstituted, advantageously it is optionally substituted with 1 to 4 substituents independently selected from:
   a halogen atom,
   a hydroxyl,
   an oxo group,
   a C₁-C₆ alkoxyl,
   a C₁-C₆ alkyl, wherein 1 or several hydrogen atoms is (are) optionally replaced with a fluorine atom, and
   a C₆-C₁₀ aryl optionally substituted with a halogen atom and/or a C₁-C₆ alkyl, preferably a fluorophenyl (in particular 4-fluorophenyl); and
   a C₃-C₆ cycloakyl optionally substituted with a C(O)NHR or NHC(O)R group wherein R represents a C₆-C₁₀ aryl optionally substituted with a halogen atom and/or a C₁-C₆ alkyl, in particular a phenyl optionally substituted with a halogen atom and/or a C₁-C₆ alkyl, preferably a phenyl optionally substituted with a halogen atom, in particular it is substituted cyclopropyl with the formula

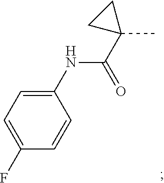

Cy represents a phenyl or a 5-10-membered heteroaryl containing from 1 to 3 heteroatoms independently selected from N, O and S, said heteroaryl optionally being substituted with an oxo group,
a pharmaceutically acceptable salt thereof or a mixture thereof,
for use in the prevention and/or treatment of viral infections.

The compound of formula (I) according to the invention may be in the form of a stereoisomer or a mixture of stereoisomers, such as tautomers, enantiomers or diastereoisomers.

In a particular embodiment of the invention, the compound of the invention is of formula (Ia):

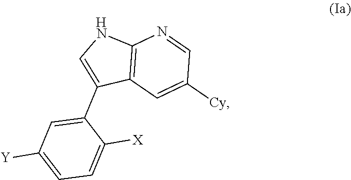

wherein X, Y and Cy are as defined above and below.

In a particular embodiment, X represents a halogen, in particular fluorine.

In a particular embodiment, X represents a hydrogen.

Cy preferably represents a phenyl, furan, pyrrole, imidazole, pyrazole, 3-oxo-2,3-dihydro-1H-pyrazole, thiophene, pyridine, 2(1H)-pyridinone, 4(1H)-pyridinone, pyrimidine, pyrimidine-2,4-dione, benzofuran, benzodioxole, indole or benzimidazole. In some embodiments, Cy represents a phenyl or a monocyclic 5-7-membered heteroaryl containing from 1 to 3 heteroatoms independently selected from N, O or S. Thus, even more preferably, Cy represents a phenyl, thiophene, furan, pyridine or pyrimidine. Even more preferably, Cy represents a phenyl, thiophene, pyridine or pyrimidine. In particular, Cy represents a phenyl or a thiophene.

In a particular embodiment, Y represents —CH═NOH, CH₂NH₂, aryl or heteroaryl, optionally mono- or polysubstituted.

In another particular embodiment, Y represents —CH₂OH.

In another particular embodiment, Y represents -L-(CH₂)p-W, with L, p and W being as defined above or below.

For example, when L represents —CH₂NH—, —CH═N—, or —CH₂N═CH—, preferably —CH₂NH—, p is between 0 and 2, preferably equal to 1 or 2, preferably 1, and W is advantageously selected from:
a phenyl optionally substituted with 1 or 2 groups independently selected from a bromine atom, a fluorine atom, a hydroxyl, a methoxy, a trifluoromethyl, and a triazole, preferably from a bromine atom, a fluorine atom, a hydroxyl, a methoxy and a 1,2,4-triazole, and
a heteroaryl selected from a furan, pyrrole, imidazole, pyrazole, thiophene, pyridine, pyrimidine, benzofuran, benzodioxole, indole and benzimidazole, preferably chosen from indole, pyridine, imidazole, benzimidazole, benzofuran, benzodioxole and pyrazole, said heteroaryl being optionally substituted with 1 to 4 groups independently selected from: a halogen atom (especially fluorine or bromine), an oxo group, a hydroxyl, a methoxy, a methyl, a trifluoromethyl, a phenyl and a fluorophenyl, in particular chosen from a methyl, a phenyl and 4-fluorophenyl.

In particular, in this embodiment, W can be chosen from a furan, pyrrole, imidazole, pyrazole, 3-oxo-2,3-dihydro-1H-pyrazole, thiophene, pyridine, 2(1H)-pyridinone, 4(1H)-pyridinone, pyrimidine, pyrimidine-2,4-dione, benzofuran, benzodioxole, indole, benzimidazole, optionally substituted with 1 to 4 groups independently selected from methyl, phenyl and 4-fluorophenyl.

In particular, when L represents CH₂NH, CH═N, or CH₂N═CH, preferably CH₂NH, p is preferably equal to 1 or 2, in particular 1, and W is then preferably chosen from:

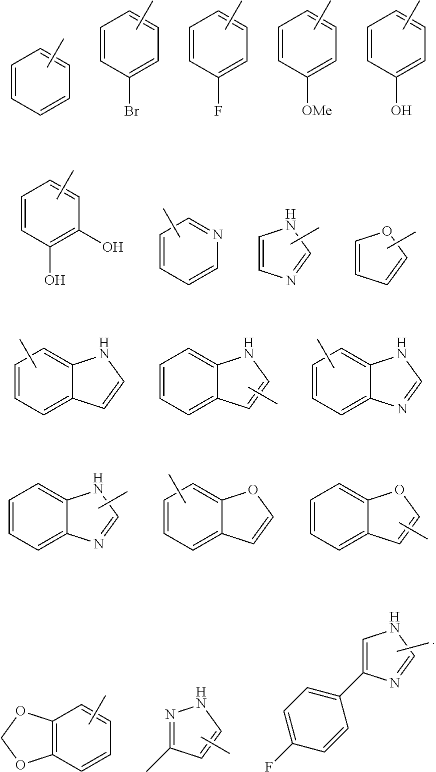

Even more preferably, W is an imidazole, in particular an unsubstituted imidazole.

In the embodiment where L represents —CH₂NH—, —CH═N—, or —CH₂N═CH—, preferably —CH₂NH—, X preferably represents a halogen, in particular fluorine, and Cy advantageously represents a phenyl or a monocyclic 5-7-membered heteroaryl containing from 1 to 3 heteroatoms independently selected from N, O or S, in particular a thiophene.

Furthermore, when L represents —C(O)NH— or —NHC(O), preferably —NHC(O)—, p is between 0 and 2, preferably equal to 0 or 1, in particular 0, and W is advantageously chosen from:
a phenyl or naphthalene optionally mono- or polysubstituted, advantageously with a halogen atom, a hydroxyl, a C₁-C₆ alkyl, or a C₁-C₆ alkoxyl, preferably a fluorine atom, a hydroxyl, a methoxy or a trifluoromethyl, even more preferably a hydroxyl,
a heteroaryl selected from a furan, pyrrole, imidazole, pyrazole, thiophene, pyridine, pyrimidine, benzofuran, benzodioxole, indole and benzimidazole, said heteroaryl being optionally substituted with 1 to 4 substituents independently selected from:
a halogen atom, in particular fluorine,
a hydroxyl,
an oxo group,
a methoxy,
a methyl or trifluoromethyl, and
a C₆-C₁₀ aryl optionally substituted with a halogen atom and/or a C₁-C₆ alkyl, preferably a fluorophenyl (in particular 4-fluorophenyl), and
a C₃-C₆ cycloakyl optionally substituted with a —C(O)NHR or —NHC(O)R group wherein R represents a C₆-C₁₀ aryl optionally substituted with a halogen atom and/or a C₁-C₆ alkyl, in particular the substituted cyclopropyl with the formula

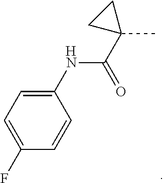

In particular, in this embodiment, W can be chosen from a furan, pyrrole, imidazole, pyrazole, 3-oxo-2,3-dihydro-1H-pyrazole, thiophene, pyridine, 2(1H)-pyridinone, 4(1H)-pyridinone, pyrimidine, pyrimidine-2,4-dione, benzofuran, benzodioxole, indole, benzimidazole, optionally substituted with 1 to 4 selected from:
a halogen atom, in particular fluorine,
a methyl, and
a C₆-C₁₀ aryl optionally substituted with a halogen atom, preferably a fluorophenyl, in particular 4-fluorophenyl.

Advantageously, when L represents —C(O)NH— or —NHC(O), preferably —NHC(O)—, p is preferably equal to 0 or 1, in particular 0, and W is advantageously chosen from:
2(1H)-pyridinone, optionally substituted with 1 to 4 substituents, in particular 1 to 2, independently selected from:
a C₆-C₁₀ aryl optionally substituted with a halogen atom, preferably a fluorophenyl, in particular 4-fluorophenyl, and
a methyl, and
a C₃-C₆ cycloakyl optionally substituted with a —C(O)NHR or —NHC(O)R group wherein R represents a C₆-C₁₀ aryl, in particular a phenyl, optionally substituted with a halogen atom, in particular substituted cyclopropyl of formula

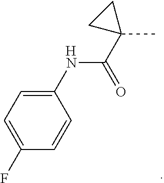

In particular, when L represents C(O)NH— or —NHC(O)—, preferably —NHC(O)—), p is preferably equal to 0 or 1, in particular 0, and W is advantageously chosen from:

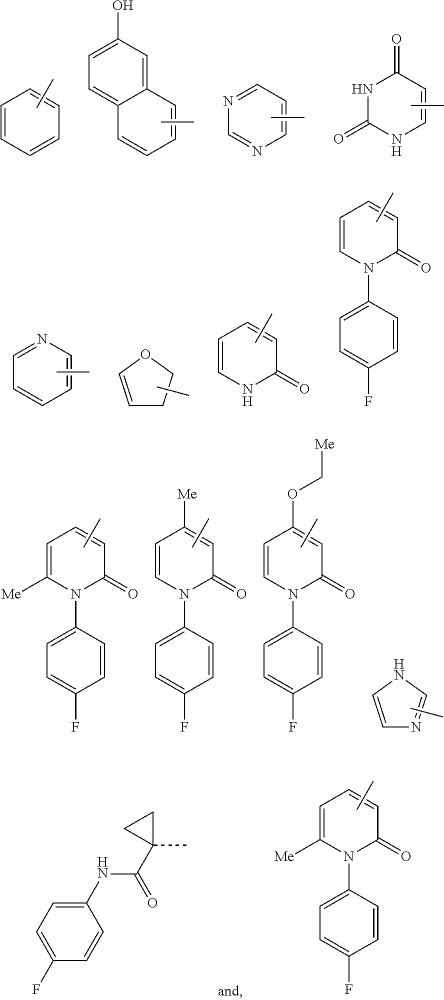

preferably

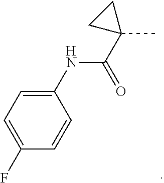

In the embodiment where L represents —C(O)NH— or —NHC(O), preferably —NHC(O), X represents a hydrogen atom or a halogen atom, in particular fluorine, and Cy advantageously represents a phenyl.

Preferably, the compound is chosen from:

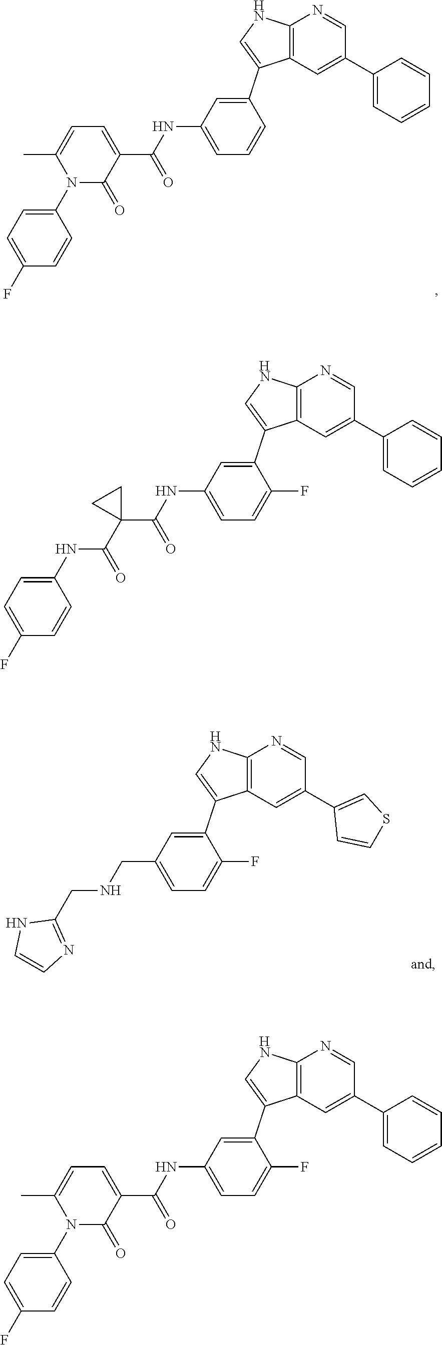

preferably

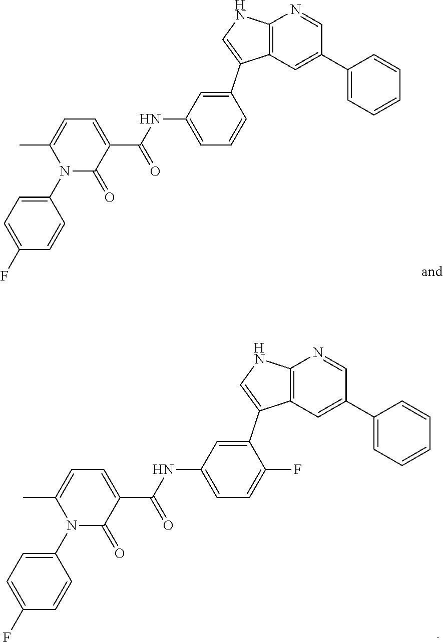

### COMPOSITIONS AND THERAPEUTIC APPLICATIONS

The compounds of the present invention inhibit AXL receptors. As AXL is involved in numerous biological processes and is a therapeutically validated target, the compounds of the present invention and their pharmaceutically acceptable salts, or the compositions of the invention as defined below, are useful as a medicament, in particular in the treatment of viral infections, the infectious cycle of which depends on AXL and the associated signaling.

The present invention relates to compounds of formula (I) as defined above for use in the prevention and/or treatment of viral infections.

More particularly, the compounds may be used to prepare pharmaceutical compositions comprising, as an active ingredient, at least one compound of formula (I) described above or a pharmaceutically acceptable salt thereof, with at least one pharmaceutically acceptable excipient. Thus, the present invention relates to a pharmaceutical composition comprising a compound of formula (I) according to the invention or a pharmaceutically acceptable salt thereof as an active ingredient, and a pharmaceutically acceptable excipient, for use in the prevention and/or treatment of viral infections. Said excipients are chosen according to the pharmaceutical form and the mode of administration desired from the usual excipients which are known to the person skilled in the art.

The pharmaceutical composition according to the invention may further comprise an additional therapeutic agent, typically chosen from an antiviral agent, an anti-inflammatory agent, an immunomodulatory or immunosuppressive agent, an agent for the treatment of immunodeficiency disorders and an agent for pain treatment. In particular, it is an agent useful in a treatment against viral infections.

One object of the invention therefore relates to the use of the compounds of formula (I) as defined above or of a pharmaceutical composition as defined above in the prevention and/or treatment of viral infections.

In other words, the invention relates to the use of a compound of formula (I) as defined above or of a pharmaceutical composition as defined above for the preparation of a medicament for the prevention and/or treatment of viral infections.

The compounds of formula (I) according to the invention or the pharmaceutically acceptable salts thereof, or the pharmaceutical composition according to the invention are particularly useful for the prevention and/or treatment of viral infections due to Flaviviruses, such as dengue virus, Zika virus, West Nile virus, Kunjin virus, Alphaviruses, such as Chikungunya virus, Mayaro virus, Semliki Forest virus, Sindbis virus, Eastern equine encephalitis virus, Western equine encephalitis virus, Venezuelan equine encephalitis virus, Filoviruses such as Ebola virus, Marburg fever virus, arenaviruses such as Lassa fever virus, Junin virus, Amapari virus, picornaviruses such as vesicular stomatitis virus, paramyxoviruses such as influenza virus, or coronaviruses such as SARS-COV2.

In a particular embodiment, the invention relates to the compounds of formula (I) as defined above or the pharmaceutical composition as defined above in the prevention and/or treatment of viral infections due to coronavirus, in particular SARS-Cov2.

The present invention also relates to a kit comprising:
a) a first composition comprising a compound according to the invention or a pharmaceutically acceptable salt thereof as an active ingredient, and a pharmaceutically acceptable excipient, and
b) a second composition comprising an additional therapeutic agent, typically selected from an antiviral agent, an anti-inflammatory agent, an immunomodulatory or immunosuppressive agent, an agent for treating immunodeficiency disorders and an agent for pain treatment, preferably an agent useful in treatment against viral infections, as a combination product for separate, concomitant or sequential use.

Said kit is useful as a medicament, in particular for the prevention and/or treatment of viral infections, in particular viral infections as defined above.

The pharmaceutical compositions according to the invention can be administered parenterally, such as by an intravenous or intradermal route, or topically, orally or nasally.

Forms that can be administered parenterally include aqueous suspensions, isotonic saline solutions or sterile and injectable solutions which may contain pharmacologically compatible dispersion and/or wetting agents. Forms that can be administered orally include tablets, soft or hard gel capsules, powders, granules, oral solutions and suspensions. Forms that can be administered nasally include aerosols. Forms that can be administered topically include patches, gels, creams, ointments, lotions, sprays, and eye drops.

Preferably, the compounds or compositions of the invention are administered orally or parenterally (in particular intravenously).

The effective dose of a compound of the invention varies as a function of numerous parameters such as, for example, the chosen administration route, the weight, the age, the sex, the state of progress of the pathology to be treated and the sensitivity of the individual to be treated.

The present invention, according to another of its aspects, also relates to a method for preventing and/or treating the pathologies indicated above which comprises the administration, to a patient in need thereof, of an effective dose of a compound according to the invention, or a pharmaceutically acceptable salt thereof or of a composition according to the invention, preferably parenterally (in particular intravenously) or orally.

### METHOD FOR PREPARING THE COMPOUNDS OF THE INVENTION

The present invention also relates to methods for preparing the compounds described above, in particular from 5-bromo-3-iodo- 1H-pyrrolo[2,3-b]pyridine (II).

According to the first embodiment, the method concerning the invention is shown in diagram 1.

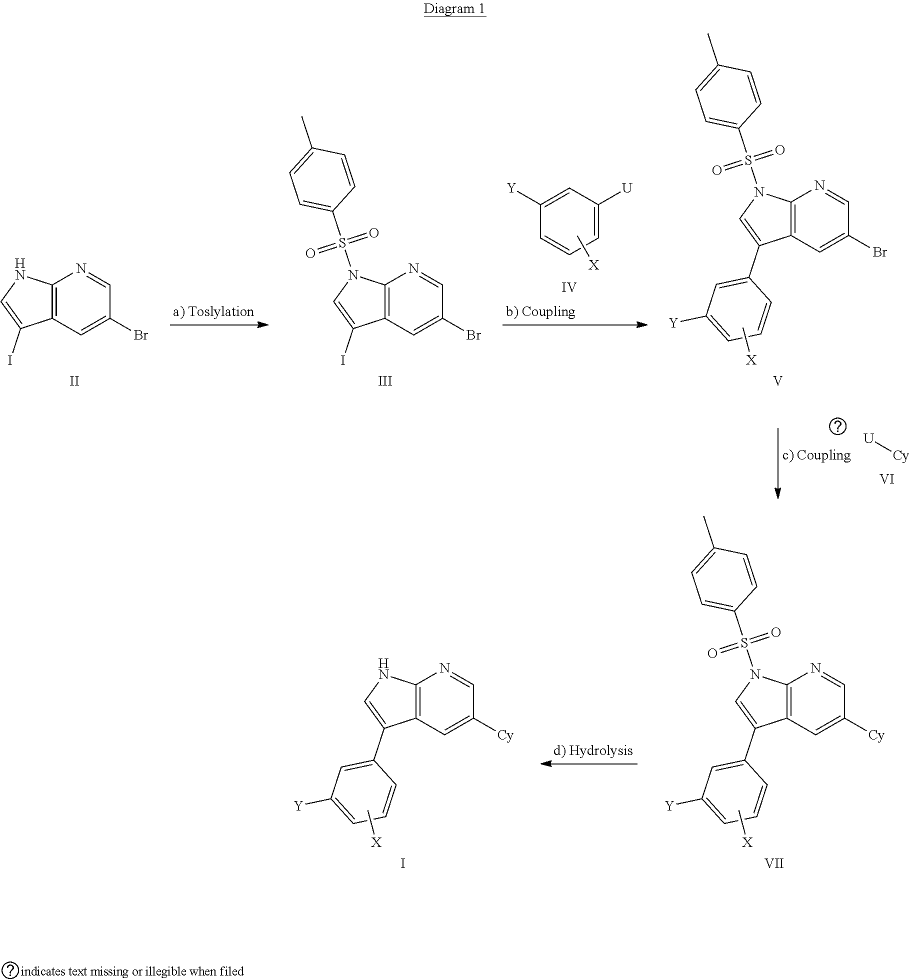

where X and Cy are as defined above and Y is a group from —CHO, —CN, —CH₂OH, —CO₂H or —NO₂.

The method comprises at least the steps of:
a) tosylation of 5-bromo-3-iodo-1H-pyrrolo[2,3-b]pyridine (II), for example with toluene-4-sulfonyl chloride in the presence of a base such as sodium hydride, to obtain intermediate (III),
b) coupling reaction of the Suzuki-Miyaura type in the presence of a palladium catalyst such as palladium dichloro [1,1′-Bis(diphenylphosphino)ferrocene] (Pd (dppf)Cl₂), with the intermediate of formula (IV) wherein U represents a boronic acid or its pinacol ester, to obtain the compound of formula (V),
c) coupling reaction of the Suzuki-Miyaura type in the presence of a palladium catalyst such as palladium dichloro [1,1′-Bis(diphenylphosphino)ferrocene] (Pd(dppf)Cl₂), with the intermediate of formula (VI) wherein U′ represents a boronic acid or its pinacol ester, to obtain the intermediate of formula (VII), and
d) hydrolysis of the tosyl group by a base, preferably sodium hydroxide or cesium carbonate, to obtain a compound of formula (I).

The general synthesis of the intermediate amine compounds (VIII) is represented in diagram 2.

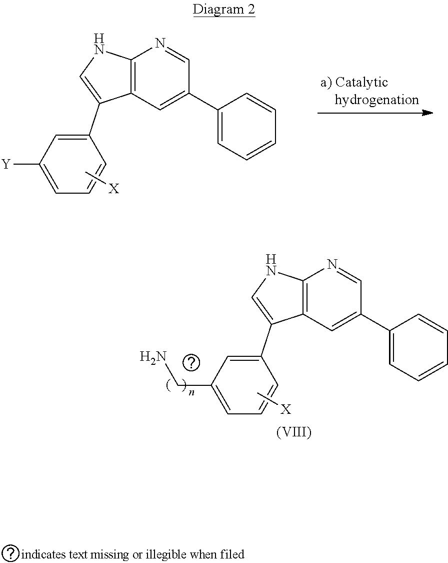

where X is as defined above and Y is a cyano or nitro group with n equal to 0 or 1.

The method comprises at least the step of:
e) catalytic hydrogenation of the nitro or cyano functions in the presence of a catalyst, for example palladium on Raney nickel or carbon, and hydrogen for example under hydrogen pressure or released in situ by ammonium formate under microwave irradiation

The person skilled in the art will naturally apply all the other synthetic techniques that have been properly described and known to synthesize these types of compounds.

The compounds of formula (I) wherein Y represents a L-(CH₂)ₚ-W group with L representing an amide group —NHCO—, are for example obtained by a method of synthesis from the amino-7-azaindole derivatives represented in diagram 3:

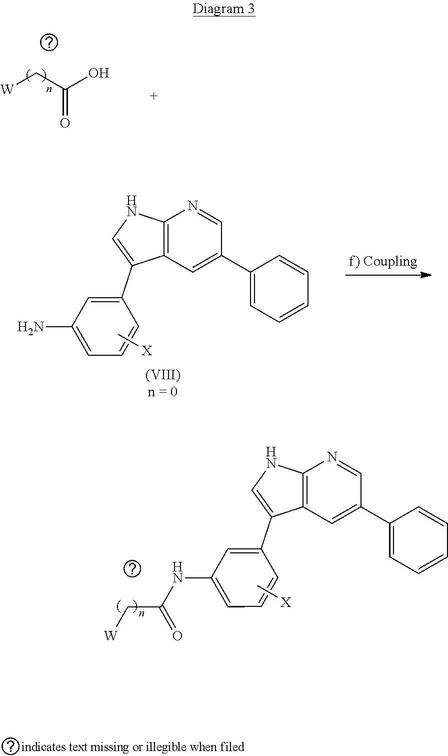

where W, X and p are as defined above.

The method of diagram 3 comprises at least one step of reaction of peptide coupling between an acid of formula W—(CH₂)ₚ—C(O)OH and the amino intermediate of formula (VIII) as defined above, in particular in the presence of at least one activating agent such as 2-(7-aza-1H-benzotriazol-1-yl)-N,N,N′,N′-tetramethyluronium (HATU) and a base such as diisopropylethylamine (DIEA).

The person skilled in the art will naturally apply all the other well-known synthetic techniques to obtain these types of amide compounds.

The compounds of formula (I) wherein Y represents a L-(CH₂)ₚ-W group with L representing a —CONH— group, are for example obtained by a method of synthesis from carboxylic 7-azaindole derivatives of formula (IX) represented in diagram 4 according to two methods among others:

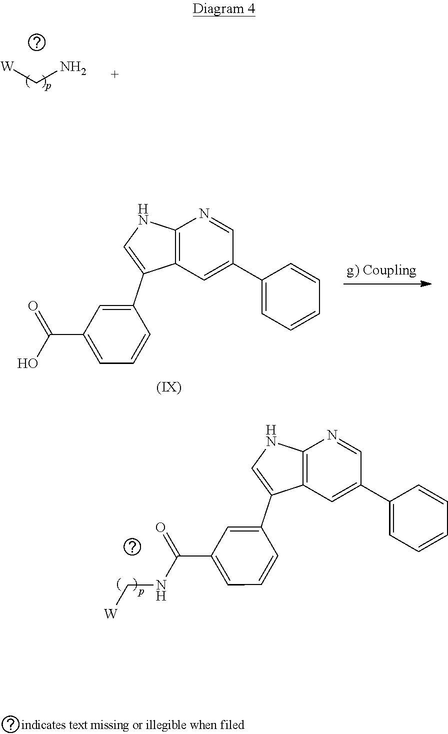

where W and p are as defined above.

Advantageously, the methods of diagram 4 comprise at least one step of peptide coupling reaction between an acid of formula (IX) and an amine of formula W-(CH₂)ₚ—NH₂, either by coupling or by in situ generating of acyl chlorides by prior action of thionyl chloride.

The person skilled in the art will naturally apply all the other well-known synthetic techniques to obtain these types of amide compounds.

According to another embodiment, concerning the method of synthesis of the secondary imine or amine compounds of the present invention, that is the compounds of formula (I) wherein Y represents a group L-(CH₂)ₚ-W with L representing a group —CH═N— or —CH₂NH—, three methods among others are represented in diagram 5:

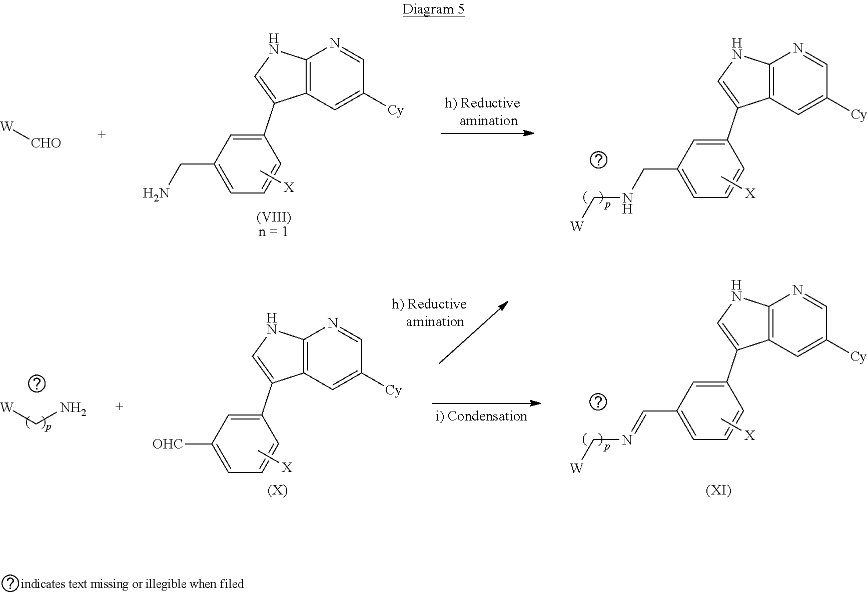

where W, X, Cy, and p are as defined above.

Advantageously, the methods of diagram 5 comprise at least one step of reductive amination between either a compound of formula (VIII) wherein n is equal to 1, with aldehydes of formula W—CHO, or between the 7-azaindole aldehydes of formula (X) and an amine of formula W—(CH₂)ₚ—NH₂.

The methods of diagram 5 also comprise a condensation reaction between the 7-azindole aldehydes compounds of formula (X) and an amine of formulae W—(CH₂)ₚ—NH₂ to form the imine compounds of formula (XI).

The aldehydes of formula W—CHO and the amines of formula W—(CH₂)ₚ—NH₂ are in particular commercially available, or easily obtained according to preparation methods known by the person skilled in the art.

The person skilled in the art will naturally apply all the other well-known synthetic techniques to obtain these types of imine and amine compounds.

A method for synthesizing the phenol compounds according to the present invention is represented in diagram 6 below:

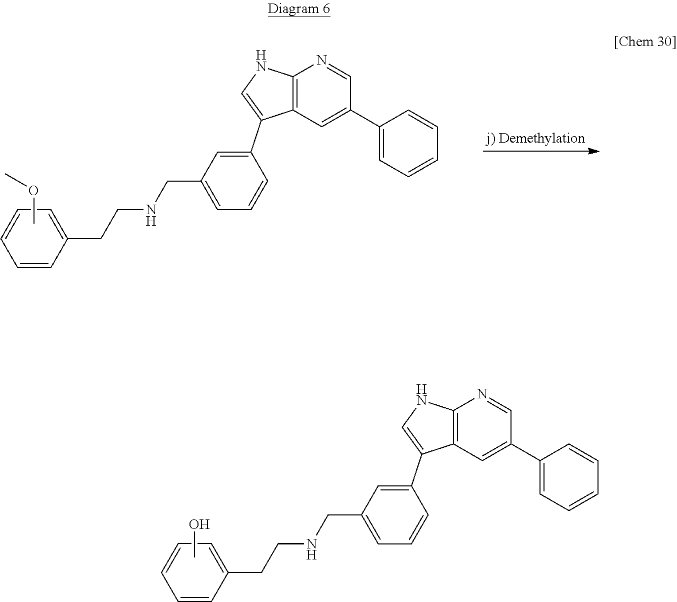

Advantageously, the method comprises at least the following step:
j) demethylation of the methoxyphenyl compound in the presence of boron tribromide (BBr₃) to form the desired hydroxyphenyl.

The person skilled in the art will naturally apply all other well-known synthetic techniques to synthesize the desired hydroxyphenyls.

### DESCRIPTION OF THE FIGURES

FIG. 1: Anti-ZIKV activity of compound 4. The cells were preincubated for 1 hour with the compounds at the indicated concentrations (0.1, 0.2, 0.4, 0.5, 1 μM), infected with the viral strain ZIKV BeH8 (MOI=0.1), and kept in culture for 24 hours in the presence of the compounds. The residual viral replication is determined by quantification of the viral genomic RNA present in the cells, by qRT-PCR. The values are expressed as a percentage of the infection detected in the control condition obtained by incubation of cells in the presence of DMSO (0), the dilution solvent of the compounds. The values are averages of triplicates+standard deviation: a) histogram representation, b) logarithmic representation.

FIG. 2: Anti-KUNV activity of compounds 4 and 15. The cells were preincubated for 1 hour with the compounds at the indicated concentrations (0.001, 0.1, 0.5, 1 and 2.5 μM), then infected with a KUNV-luciferase reporter virus (MOI=0.1). The cells are kept in culture for 24 hours in the presence of the compounds. Residual viral replication, measured by quantification of luciferase activity in the cell lysate using the reagent Genofax A (Yelen) is expressed as a percentage of the infection detected in the control condition obtained by incubation of cells in the presence of DMSO (0), the dilution solvent of the compounds. The values are averages of triplicates+standard deviation.

FIG. 3: Anti-CHIKV activity of compound 4. The cells were preincubated for 1 hour with the compound at the indicated concentrations (0.001, 0.1, 0.25, 0.5, 1 and 1.5 μM), and then infected with the viral strain CHIKV LR-OPY1 containing a luciferase reporter gene (MOI=0.1). The cells were kept in culture for 24 hours in the presence of the compound. Residual viral replication, measured by quantification of luciferase activity in the cell lysate (reagent Genofax Yelen) is expressed as a percentage of the infection detected in the control condition obtained by incubation of cells in the presence of DMSO (0), the dilution solvent of the compound. The values are averages of triplicates+standard deviation.

FIG. 4: Anti-MAYV activity of compound 4. The cells were preincubated for 1 hour with the compounds at the indicated concentrations (0.25, 0.5, 1 and 1.5 μM), then infected with the viral strain TRVL4576-luc (MOI=0.1). The cells were kept in culture for 24 hours in the presence of the compound. Residual viral replication, measured by quantification of luciferase activity in the cell lysate (reagent Genofax A, Yelen) is expressed as a percentage of the infection detected in the control condition obtained by incubation of cells in the presence of the dilution solvent of the compounds, DMSO (0). The values are averages of triplicates+standard deviation.

FIG. 5: Anti-SARS-COV2 activity of compound 5 determined in the VeroE6 line. The cells were preincubated for 1 hour with compound 5 at a concentration of 1.25 μM, then infected with the viral strain (MOI=0.1). The cells were kept in culture for 24 hours in the presence of the compound. The residual viral replication is measured by quantification of the viral RNA in the cell culture by qRT-PCR. The control condition is obtained by incubation of cells in the presence of DMSO (0). R428/Bemcentinib is evaluated in parallel. The values are averages of triplicates+standard deviation.

### EXAMPLES

The invention will be better understood upon reading the following examples, which are given purely by way of illustration and should not be interpreted as limiting the scope of the present invention.

### Example 1. Synthesis

### Material

The syntheses and analyses were carried out under the following conditions.

Magnetic Nuclear Resonance ¹H and ¹³C:

Apparatus: Bruker Avance 400 (400 MHZ); Bruker Avance 300 (300 MHZ)

Conditions of use: Ambient temperature, chemical shifts expressed in parts per million (ppm), multiplicity of signals indicated by lowercase letters (singlet s, doublet d, triplet t, quadruplet q, multiplet m), dimethyl sulphoxide d₆, methanol d₄, chloroform d₁ as deuterated solvents.

### High-Pressure Liquid Chromatography (HPLC):

Apparatus: Agilent Technology 1260 Infinity

Conditions of use: Zorbax SB-C18 column or Eclipse plus (2.1×50 mm), 1.8 μm; temperature: 30° C., flow rate: 0.5 mL/min for the methods X and Y and 0.4 mL/min for the method Z, elution gradient Water (A)/Acetonitrile (B)/Formic acid 0.1% (Time (min)/% B):
Method X: 0/10, 0.3/10, 5.7/100, 6.0/100
Method Y: 0/10, 1/50, 6/100, 8/100
Method Z: 0/10, 11/100, 15/100

### Mass Spectrometry (MS):

Apparatus: Quadripole Agilent Technologies 6120

Conditions of use: ElectroSpray (ESI) in positive and/or negative mode.

### Weighing:

Appliance: Denver Instrument TP214 (0.1 mg precision)

Conditions of use: Weighing carried out to the nearest milligram.

Pressure reactions:

Apparatus: Autoclave Parr 300 mL.

Conditions of use: Hydrogenation under 20 bars of hydrogen.

Reaction under microwave irradiation:

Appliance: CEM Discover SP®

Conditions of use: Power of 200 Watts, Average stirring speed

In the following, the following abbreviations are used:

| | | | |
|---|---|---|---|
| eq | equivalent | DMF | N,N-dimethylformamide |
| RT | Room Temperature | DMSO | dimethylsulfoxide |

### General synthesis of 3,5-diaryl-1H-pyrrolo[2,3-b]pyridines (I) Intermediates

### 1^{st} Step, Synthesis of 5-bromo-3-iodo-1-(toluene-4-sulfonyl)-1 H-pyrrolo[2,3-b]pyridine (III)

3 g of 5-bromo-3-iodo-1 H-pyrrolo[2,3-b]pyridine (II) are diluted in 60 mL of anhydrous THF under argon and cooled in an ice bath. 558 mg (1.5 eq) of sodium hydride (60%) are added slowly and the medium is stirred at 0° C. for 20 minutes. Then tosyl chloride (2.11 g, 1.2 eq) is added, and the medium is stirred at RT for 5 hours. The solvent is evaporated. The residue obtained is suspended in a minimum of petroleum ethers and filtered. The precipitate is washed twice with 2M sodium hydroxide solution, then dried under vacuum overnight.

RMN ¹H (400 MHZ, DMSO-d6) δ (ppm) 8.52 (d, J=1.9, 1H), 8.22 (s, 1H), 8.01 (m, 3H), 7.44 (d, J=8.2, 2H), 2.51 (s, 3H).

Yield: 97%; HPLC: 99%; MS [M+1]: 476.9-478.9

### 2^{nd} Step, General Synthesis of 3-5-bis-aryl-1 H-pyrrolo[2,3-b]pyridines

Method 1: In a schlenck under argon, 5-bromo-3-iodo-1-(toluene-4-sulfonyl)-1H-pyrrolo[2,3-b]pyridine (100 mg), the first boronic acid to be coupled (1 eq) and K2CO3 (3 eq) are, suspended in a dioxane/water mixture (9/1, 3 mL), the mixture is degassed under argon for 20 minutes. Pd(dppf)Cl₂ (2%) is added and the mixture is stirred for 5 hours under reflux. After checking the completion of the coupling reaction by LC/MS analysis, the second boronic acid (1.2 eq) and K2CO3 (3 eq) are added to the medium which is degassed again under argon for 20 minutes. Pd(dppf)Cl₂ (2%) is then added and the mixture is stirred under reflux overnight. The solvents are evaporated, the residue is taken up in ethyl acetate. The organic phase is washed with an aqueous solution saturated with NaHCO3 and then saturated aqueous NaCl solution, dried over anhydrous Na2SO4, filtered and evaporated. The medium is taken up in a mixture of tetrahydrofuran/methanol (1/1, 6 mL) and stirred for 3 hours at RT with cesium carbonate (3 eq). The solvents are evaporated off, the residue is taken up in ethyl acetate, washed with an aqueous solution saturated with NaHCO3, and then an aqueous solution saturated with NaCl, dried over Na2SO4, filtered and evaporated to dryness, and then purified on a reverse phase silica column (water/acetonitrile) with 1% trifluoroacetic acid. The compound is isolated after neutralization with NaHCO3.

Method 2: 5-bromo-3-iodo-1-(toluene-4-sulfonyl)-1 H-pyrrolo[2,3-b]pyridine (III) (100 mg), the first boronic acid to be coupled (IV) (1 eq) and potassium carbonate (3 eq) are suspended in a dioxane/water (3/1, 3 mL) mixture, and the mixture is degassed under argon for 20 minutes. Pd(dppf)Cl₂ (2%) is added and the mixture is stirred at 80° C. under microwave irradiation for 1 to 3 times 20 minutes until the starting reagent is completely consumed. The reaction medium is washed with an aqueous solution saturated with NaHCO3 and extracted with ethyl acetate. The solvents are evaporated off, the residue is taken up in ethyl acetate, washed with an aqueous solution saturated with NaHCO3, dried over Na2SO4, filtered and evaporated to dryness, and then purified on a silica column. The compound is then dissolved in a dioxane/water (3/1, 4 mL) mixture, in the presence of the second boronic acid or its ester of pinacol (VI) (1.2 eq) and K2CO3 (3 eq). The medium is degassed again under argon for 20 minutes, Pd(dppf)Cl₂ (0.025 eq) is then added and the mixture is stirred at 120° C. for 45 minutes under microwave irradiation. 500 μL of 1 M sodium hydroxide (3 eq) are added to the reaction medium and stirred at 100° C. for 45 minutes under microwave irradiation. The reaction medium is washed with an aqueous solution saturated with NaHCO3, extracted with ethyl acetate, dried over anhydrous Na2SO4, filtered and evaporated to dryness, and then purified on a normal phase silica column (dichloromethane/methanol/aqueous ammonia).

| | |
|---|---|
| 4-Fluoro-3-(5-thiophen-3-yl-1H- pyrrolo[2,3-b]pyridin-3-yl)-benzaldehyde 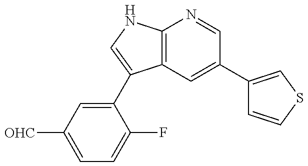 [SMILES: O=Cc1ccc(F)c(-c2c[nH]c3ncc(-c4ccsc4)cc23)c1] | Reagents: First, 2-Fluoro-5- formylbenzeneboronic acid then 3- thienylboronic acid according to method 2 Yield: 75%; HPLC: 97%; MS [M + 1]: 323.1 |
| 3-(3-Nitro-phenyl)-5-phenyl-1H- pyrrolo[2,3-b]pyridine 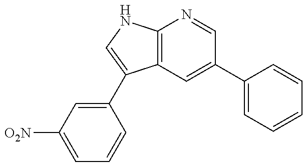 [SMILES: O=[N+]([O-])c1cccc(-c2c[nH]c3ncc(-c4ccccc4)cc23)c1] | Reagents: First, 3- nitrophenylboronic acid and then phenylboronic acid according to method 2 Yield: 98%; HPLC: 94%; MS [M + 1]: 316.1 |
| 3-(2-Fluoro-5-nitro-phenyl)-5-phyenyl-1H- pyrrolo[2,3-b]pyridine 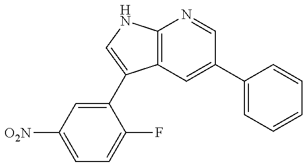 [SMILES: O=[N+]([O-])c1ccc(F)c(-c2c[nH]c3ncc(-c4ccccc4)cc23)c1] | Reagents: First, the 2-(2-Fluoro-5- nitrophenyl)-4,4,5,5-tetramethyl- [1,3,2]dioxaborolane and then phenylboronic acid according to method 2 Yield: 52%; HPLC: 96%; MS [M + 1]: 334.1 |

### Synthesis of the Amines

Method 1: Synthesis of amines by reduction of corresponding nitro compounds.

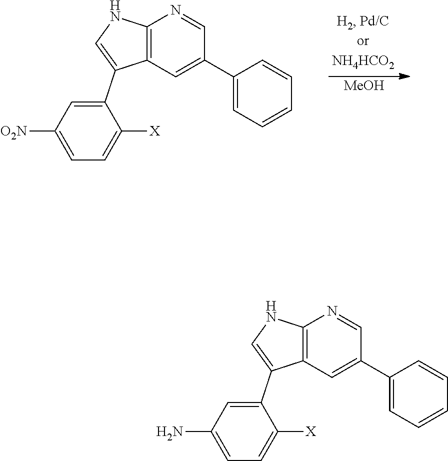

Protocol 1: The nitro derivative is placed in 200 ml of methanol in an autoclave. 10% by mass of 10% palladium-on-carbon is added under argon. The autoclave is closed, purged with Argon, and placed under 30 bar of hydrogen. The medium is stirred at room temperature overnight. The autoclave is then emptied, purged with argon. The medium is filtered through celite and then washed with methanol. The filtrate is evaporated, taken up in ethyl acetate, washed twice with an aqueous solution saturated with NaHCO3, once with an aqueous solution saturated with NaCl, dried over Na2SO4, filtered on cotton and evaporated to dryness to give the desired amine derivative.

Protocol 2: 100 mg (0.3 mmol) of nitro derivative and 189 mg (10 eq) of ammonium formate are suspended in 3 mL of methanol under argon. Suspension of 15 mg of palladium on carbon in 1 mL of methanol is then added and the whole is stirred for 30 minutes at 130° C. under microwave irradiations. The medium is filtered through celite and then washed with methanol. The filtrate is evaporated to dryness and then purified on a normal phase silica column (eluent: dichloromethane/methanol 97/3).

| | |
|---|---|
| 3-(5-Phenyl-1H-pyrrolo[2,3-b]pyridin-3-yl)- phenylamine 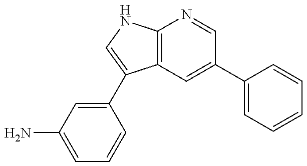 [SMILES: Nc1cccc(-c2c[nH]c3ncc(-c4ccccc4)cc23)c1] | Reagent: 3-(3-Nitro-phenyl)-5- phenyl-1H-pyrrolo[2,3-b]pyridine according to protocol 1 Yield: 72%; HPLC: 85%; MS [M + 1]: 286.2 |
| 4-Fluoro-3-(5-phenyl-1H-pyrrolo[2,3-b]pyridin-3- yl)-phenylamine 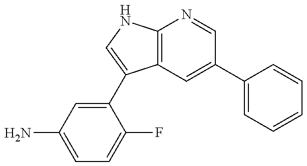 [SMILES: Nc1ccc(F)c(-c2c[nH]c3ncc(-c4ccccc4)cc23)c1] | Reagent: 3-(2-Fluoro-5-nitro- phenyl)-5-phenyl-1H- pyrrolo[2,3-b]pyridine according to protocol 2 Yield: 70%; HPLC: 98%; MS [M + 1]: 304.1 |

### Synthesis of the Various Non-Commercial Carboxylic Acids

1-(4-fluorophenyl)-6-methyl-2-oxo-1,2-dihydro-pyridine-3-carboxylic acid was obtained in accordance with the procedure described in [Flynn et al. Organic Process Research & Development (2014), 18(4), 501-510] (HPLC: 97%; MS [M+1]: 248.2).

1-(4-fluorophenyl carbamoyl)-cyclopropane carboxylic acid was obtained in accordance with the procedure described in [Zhan et al. Medicinal Chemistry Letters (2014), 5(6), 673-678] (HPLC: 100%; MS [M+1]: 224.1)

### General Synthesis of Amide Compounds from amino-7-azaindole and Carboxylic Acids Compounds

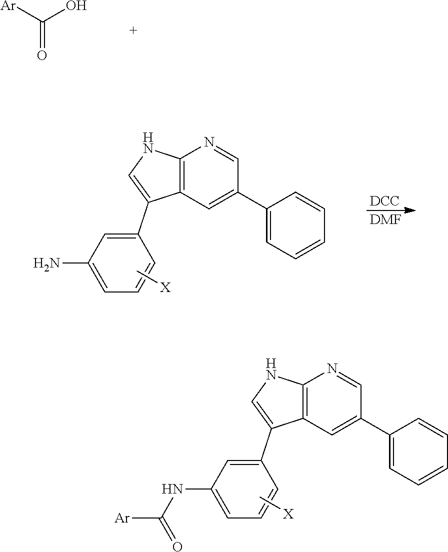

Carboxylic acid (1 eq) is dissolved in anhydrous DMF and placed in dried schlenck, under an argon atmosphere. N,N′-dicyclohexylcarbodiimide (1 eq), and then the derivative amino-7-azaindole (1 eq) are added. The medium is then stirred at 70° C. overnight. The solvent is evaporated. The residue obtained is then dissolved in ethyl acetate, washed twice with an aqueous solution saturated with NaHCO3, once with an aqueous solution saturated with NaCl, dried over anhydrous sodium sulfate, filtered on cotton, evaporated to dryness and purified on a reverse phase silica column (water/acetonitrile) with 1% of trifluoroacetic acid to give the desired amide derivative.

| | |
|---|---|
| 1-(4-fluorophenyl)-6-methyl-2-oxo-1,2-dihydro-pyridine-3-carboxylic acid [3-(5-phenyl-1H-pyrrolo[2,3-b]pyridin-3-yl)-phenyl]-amide (Example 4) 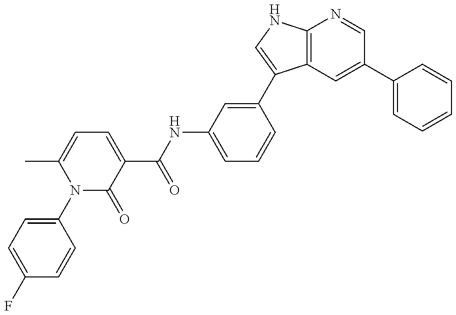 [SMILES: Cc1ccc(C(=O)Nc2cccc(-c3c[nH]c4ncc(-c5ccccc5)cc34)c2)c(=O)n1-c1ccc(F)cc1] | Reagents: 3-(5-Phenyl-1H- pyrrolo[2,3-b]pyridin-3-yl)- phenylamine and 1-(4-Fluoro- phenyl)-6-methyl-2-oxo-1,2- dihydropyridine-3-carboxylic acid Yield: 53%; HPLC: 98%; t_{R}: 5.17 min (method Y); MS [M + 1]: 515.2 |
| 1-(4-fluorophenyl)-6-methyl-2-oxo-1,2-dihydro-pyridine-3-carboxylic acid [4-fluoro-3-(5-phenyl-1H-pyrrolo[2,3-b]pyridin-3-yl)-phenyl]-amide (example 15) 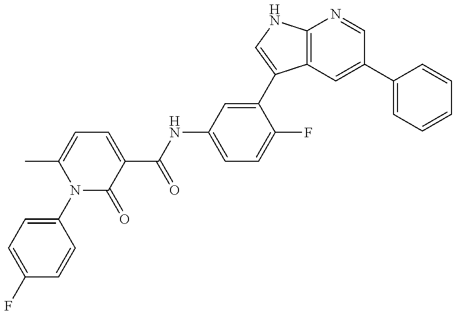 [SMILES: Cc1ccc(C(=O)Nc2ccc(F)c(-c3c[nH]c4ncc(-c5ccccc5)cc34)c2)c(=O)n1-c1ccc(F)cc1] | Reagents: 4-Fluoro-3-(5-phenyl-1H- pyrrolo[2,3-b]pyridin-3-yl)- phenylamine and 1-(4-Fluoro- phenyl)-6-methyl-2-oxo-1,2- dihydropyridine-3-carboxylic acid Yield: 38%; HPLC: 97%; t_{R}: 5.34 min (method Y); MS [M + 1]: 533.2 |
| Cyclopropane-1,1-dicarboxylic acid (4-fluoro-phenyl)-amide [3-(5-phenyl- 1H-pyrrolo[2,3-b]pyridin-3-yl)-phenyl]-amide (example 5) 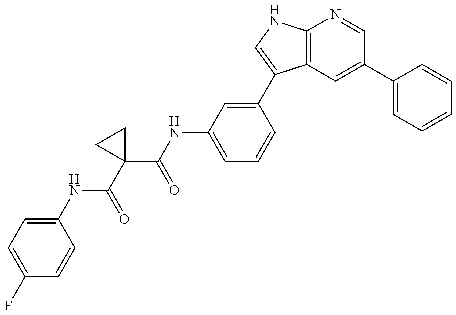 [SMILES: O=C(Nc1ccc(F)cc1)C1(C(=O)Nc2cccc(-c3c[nH]c4ncc(-c5ccccc5)cc34)c2)CC1] | Reagents: 3-(5-Phenyl-1H- pyrrolo[2,3-b]pyridin-3-yl)- phenylamine and 1-(4- fluorophenylcarbamoyl)- cyclopropane carboxylic acid Yield: 31%; HPLC: 98%; t_{R}: 5.10 min (method Y); MS [M + 1] 491.2 |

### Reductive Amination from amino-azaindoles

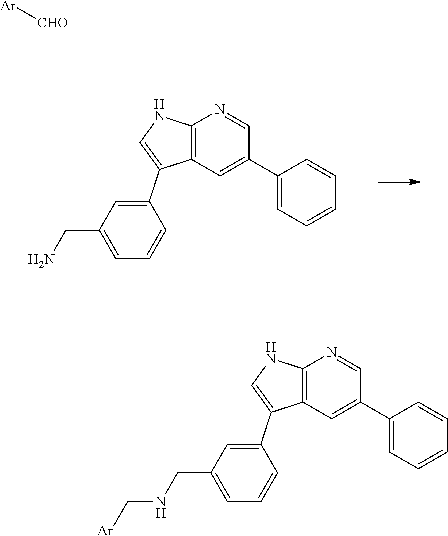

The aldehyde derivative (1 eq) and the amine derivative (1 eq) are placed in a round-bottomed flask and dissolved in a methanol/acetic acid mixture (9/1). The mixture is stirred for 3 hours at room temperature and then sodium cyanoborohydride (2 eq) is added and the medium is stirred at room temperature overnight. The solvent is evaporated off, the residue is taken up in ethyl acetate, washed twice with an aqueous solution saturated with NaHCO3, once with an aqueous solution saturated with NaCl, dried over anhydrous Na2SO4, filtered on cotton and evaporated to dryness. The residue obtained is purified on a silica column with a gradient of 100% dichloromethane to 90/10 dichloromethane/methanol-ammonia.

### Reductive Amination from formyl-azaindoles

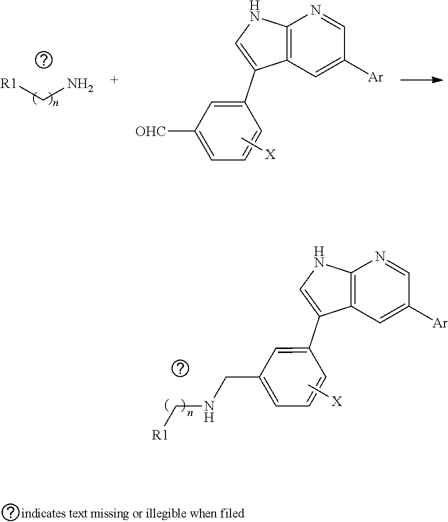

The aldehyde derivative (1 eq) and the amine derivative (1 eq) are dissolved in a methanol/acetic acid mixture (9/1). The mixture is stirred for 3 hours at room temperature and then sodium cyanoborohydride (2 eq) is added and the medium is stirred at room temperature overnight. The solvent is evaporated off, the residue is taken up in ethyl acetate, washed twice with an aqueous solution saturated with NaHCO3, once with an aqueous solution saturated with NaCl, dried over anhydrous Na2SO4, filtered on cotton and evaporated to dryness. The residue obtained is purified on a silica column with a gradient of 100% dichloromethane to 90/10 dichloromethane/methanol-ammonia.

| | |
|---|---|
| [4-Fluoro-3-(5-thiophen-2-yl-1H-pyrrolo[2,3-b]pyridin-3-yl)- benzyl]-(1H-imidazol-2-ylmethyl)-amine (example 6) 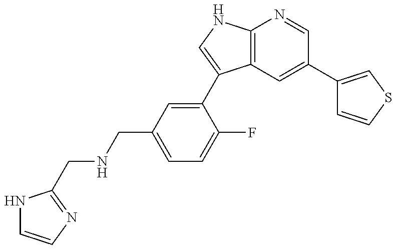 [SMILES: Fc1ccc(CNCc2ncc[nH]2)cc1-c1c[nH]c2ncc(-c3ccsc3)cc12] | Reagents: 4-Fluoro-3-(5-thiophen-3-yl- 1H-pyrrolo[2,3-b]pyridin-3-yl)- benzaldehyde and (1H-Imidazazol-2- yl)-methylamine Yield: 57%; HPLC: 94%; t_{R}: 2.80 min (method Y); MS [M + 1]: 404.1 |

### Example 2. Biological Tests

The compounds of the invention were evaluated for their antiviral activity in vitro, by testing the inhibitory effect of the molecules on the replication of infectious complete viruses. The selected cell systems (HeLa, A549, A549-ACE2 cells) are relevant for the envisaged infectious models and have been validated for the expression of the Axl molecule in flow cytometry.

### Infectious Models Used:

| Virus | Model | Strain | Cells |
|---|---|---|---|
| ZIKV | mCherry reporter | BeH-8 | HeLa |
| DENV | Luciferase reporter | DENV2 | HeLa |
| WNV | Luciferase reporter | Kunjin (KUNV) | HeLa |
| CKV | Luciferase reporter | LR OPY-1 | HeLa |
| MAYV | Luciferase reporter | TRVL4675 | HeLa |
| SARS-COV2 | Viral isolate | BetaCoV/France/IDF0371/202 | VeroE6 |

### 1—Antiviral Activity Against the Zika Virus

The tests are carried out by preincubating 2.5×10⁴ cells of the HeLa line (cervical carcinoma; ATCC #CCL-2) cultured in a 96-well plate with increasing concentrations of compounds for 1 hour at 37° C. The cells are cultured in a 5% CO2 atmosphere in a Dulbecco's modified Eagle's medium (DMEM) comprising 10% calf serum and 1% penicillin/streptomycin. The cells are then exposed to the ZIKV BeH8 strain (MOI=0.1) for 1 hour in the presence of the compound.

The viral inoculum is then removed and the cells are maintained in the presence of the compound for 24 h. The infection of the cells is determined by quantification of the viral RNAs detected by qRT-PCR using the Luna Universal One-Step RT-PCR kit. The values (DCT) are normalized relative to the quantification of the mRNA of the GAPDH. The values represented are averages of triplicates+standard deviation.

The control conditions were carried out by incubating the cells in the presence of DMSO (0), the solvent used for the solubilization of the compounds tested. The volumes of DMSO used under these conditions correspond to the volumes provided by the compounds under the test conditions.

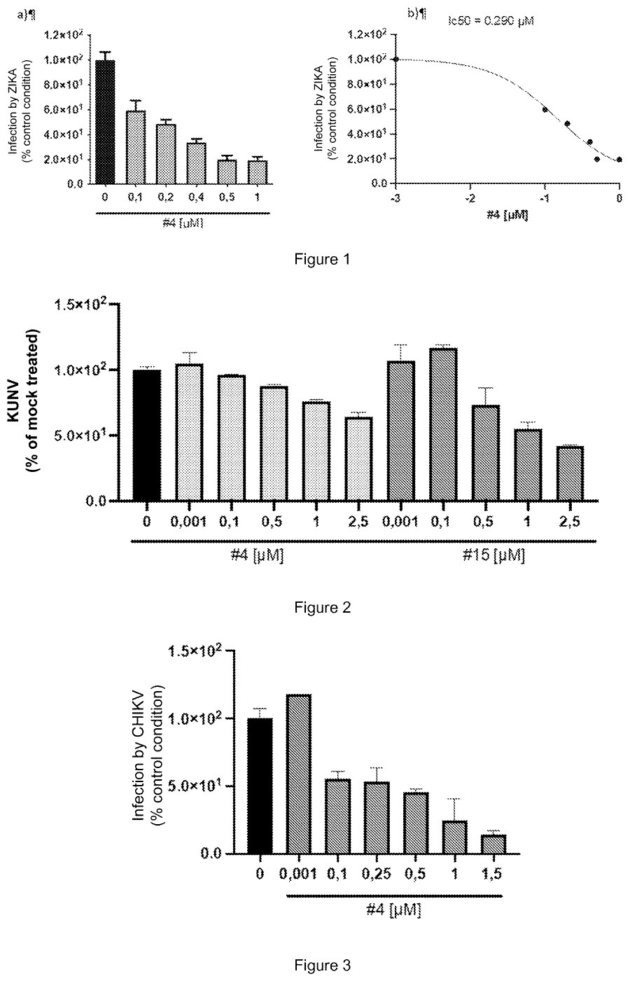

The results obtained for compound 4 are shown in FIG. 1. The IC₅₀ of compound 4 was determined using the software Graphpad Prism.

Under these experimental conditions, the compound 4 used at a concentration greater than or equal to 100 nM prevents infection of the Hela cells by the strain ZIKV BeH-8.

2—Antiviral activity against the WNV virus Kunjin strain

The tests are carried out by preincubating 4×10⁴ cells of the HeLa line (ATCC #CCL-2) cultured in a 96-well plate with increasing concentrations of compounds for 1 hour. The cells are cultivated in a 5% CO2 atmosphere in a Dulbecco's modified Eagle's medium (DMEM) comprising 10% calf serum and 1% penicillin/streptomycin. The cells are then exposed to the WNV virus, Kunjin strain expressing a nanoluciferase gene upstream of the sequence coding for the capsid protein, in the presence of the compounds, for 1 hour. The viral inoculum is then removed and the cells are maintained in the presence of the inhibitors for 24 hours.

The cells are lysed using Passive lysis Buffer (Promega) reagent. The viral infection is detected by quantification of the nanoluciferase activity in the cell lysate in the presence of the reagent Genofax A (Yelen) and using an Infinite F200PRO (Tecan) fluorimeter. The values are normalized relative to the amount of total proteins contained in the cell lysate and determined using the BCA Protein Assay kit (Pierce) by measuring the absorbance at 562 nm. The values represented are averages of triplicates+standard deviation. The control conditions are identical to those described above.

The results obtained for compounds 4 and 15 are shown in FIG. 2.

Under these experimental conditions, compounds 4 and 15 used at a concentration greater than or equal to 500 nM prevent infection of Hela cells with WNV strain Kunjin.

### 3—Antiviral Activity Against the Chikungunya Virus

The tests are carried out by preincubating 4×10⁴ cells of the HeLa line (ATCC #CCL-2) cultured in a 96-well plate with increasing concentrations of compounds for 1 hour. The cells are cultivated in a 5% CO2 atmosphere in a Dulbecco's modified Eagle's medium (DMEM) comprising 10% calf serum and 1% penicillin/streptomycin. The cells are then exposed to the CHIKV virus, LR-OPY1 strain expressing either a luciferase gene, or a sequence encoding GFP upstream of the sequence coding for the nsP3 protein. The cells are incubated for 1 hour.

The viral inoculum is then removed and the cells are maintained in the presence of inhibitors for 24 h. The viral infection is measured after lysis of the cells using the Passive lysis Buffer (Promega) reagent, either by direct quantification of the fluorescence of GFP or by quantification of the luciferase activity in the lysate of the cells carried out in the presence of the Genofax A (Yelen) reagent and using an Infinite F200PRO (Tecan) fluorimeter. The values are normalized relative to the amount of total proteins contained in the cell lysate and determined using the BCA Protein Assay kit (Pierce) by measuring the absorbance at 562 nm. In both protocols, the values are normalized relative to the amount of total proteins contained in the cell lysate and determined using the BCA Protein Assay kit (Pierce). The values represented are averages of triplicates+standard deviation. The control conditions are identical to those described above.

The results obtained for compound 4 are shown in FIG. 3.

Under these experimental conditions, the compound 4 used at a concentration greater than or equal to 100 nM prevents infection of the Hela cells by the Chikungunya virus.

### 4—Antiviral Activity Against the Mayaro Virus

The tests are carried out by preincubating 4×10⁴ cells of the HeLa line (ATCC #CCL-2) cultured in a 96-well plate with increasing concentrations of compounds for 1 hour. The cells are cultivated in a 5% CO2 atmosphere in a Dulbecco's modified Eagle's medium (DMEM) comprising 10% calf serum and 1% penicillin/streptomycin. The cells are then exposed to the MAYV virus, TRVL4576 strain expressing a luciferase gene upstream of the sequence coding for the nsP3 protein. The cells are incubated for 1 hour. The viral inoculum is then removed and the cells are maintained in the presence of inhibitors for 24 hours.

The cells are lysed using Passive lysis Buffer (Promega) reagent. The viral infection is detected by quantification of the luciferase activity in the cell lysate in the presence of the reagent Genofax A (Yelen) and using an Infinite F200PRO (Tecan) fluorimeter. The values are normalized relative to the amount of total proteins contained in the cell lysate and determined using the BCA Protein Assay kit (Pierce) by measuring the absorbance at 562 nm. In both protocols, the values are normalized relative to the amount of total proteins contained in the cell lysate and determined using the BCA Protein Assay kit (Pierce). The values represented are averages of triplicates+standard deviation. The control conditions are identical to those described above.

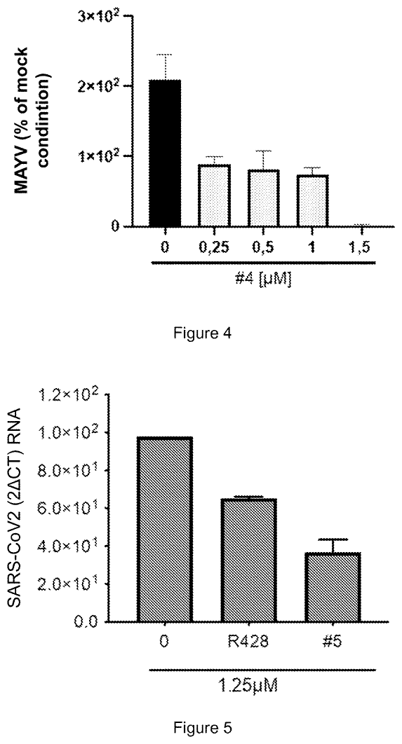

The results obtained for compound 4 are shown in FIG. 4.

For each of the tested infectious models, the IC₅₀ active compounds were determined using the software Graphpad Prism.

| Compound | CKV | MAYV | KUNV | ZIKV |
|---|---|---|---|---|
| 4 | 0.243 μM | 0.3127 μM | 1.321 μM | 0.290 μM |
| 15 | | | 0.934 μM | |
| R428/Bemcentinib | 0.239 μM | ND | ND | 0.285 μM |
| (reference) | | | | |

6—Antiviral Activity Against SARS-COV2 coronavirus

The inhibitory activity of the compounds of the invention on SARS-COV2 infection was evaluated in vitro by infection with the green monkey line VeroE6 (immortalized kidney epithelium, ATCC#CRL-1586).

Assessment of antiviral activities in VeroE6 cells

The infection tests are carried out by preincubating 4×10⁴ VeroE6 cells, cultivated in a 96-well plate, with the indicated concentrations of compounds for 1 hour. The cells are cultivated in a 5% CO2 atmosphere in a Dulbecco's modified Eagle's medium (DMEM) comprising 2% calf serum and 1% penicillin/streptomycin. The cells were then exposed to the SARS-COV2 virus, BetaCoV/France/IDF0371/2020 (MOI=0.01) for 2 hours before elimination of the inoculum, washing and regrowth of the cells in culture. 24 h after the viral challenge, the infection is quantified by amplification of the viral RNA by qRT-PCR using the Luna Universal One-Step RT-PCR kit. The values (ΔCT) are normalized relative to the quantification of the mRNA of the GAPDH. The values represented are averages of triplicates+standard deviation. The control conditions were carried out by incubating the cells in the presence of DMSO (0), the solvent used for the solubilization of the compounds tested. The volumes of DMSO used under these conditions correspond to the volumes provided by the compounds under the test conditions.

Parallel treatment, under the same experimental conditions, was carried out with the

R428/Bemcentinib, Axl inhibitors in clinical trial phase developed by the company Bergenbio.

The results obtained for compound 5 are shown in FIG. 5.

Under these experimental conditions, compound 5 reduces the infection of the cells by the SARS-COV2 virus.

1. A compound of formula (I): 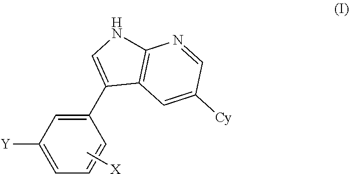
in which
X represents a hydrogen atom or a halogen atom,
Y is selected from —CH₂OH, —CH═NOH, —CH₂NH₂, an aryl optionally mono- or polysubstituted, a heteroaryl optionally mono- or polysubstituted, and -L-(CH₂)p-W,
L represents —C(O)NH—, —CH₂NH—, —CH═N—, —NHC(O)—, or —CH₂N═CH—,
p is an integer from 0 to 2,
when L represents —CH₂NH—, —CH═N—, or —CH₂N═CH—, W is selected from:
a C₁-C₆ alkyl, preferably a methyl,
a C₆-C₁₀ aryl, preferably a phenyl, optionally mono- or polysubstituted, advantageously by 1 or 2 groups independently selected from:
a halogen atom,
a hydroxyl,
a C₁-C₆ alkoxyl, preferably a methoxy,
a C₁-C₆ alkyl, wherein 1 or several hydrogen atoms is (are) optionally replaced with a fluorine atom, preferably a trifluoromethyl, and
a 5-10 membered heteroaryl comprising from 1 to 3 heteroatoms independently selected from N, O and S, in particular a 5-membered heteroaryl group comprising from 1 to 3 heteroatoms independently selected from N, O and S such as triazole, preferably 1,2,4-triazole;
a 5-10 membered heteroaryl containing from 1 to 3 heteroatoms independently selected from N, O and S, such as a furan, pyrrole, imidazole, pyrazole, thiophene, pyridine, pyrimidine, benzofuran, benzodioxole, indole, benzimidazole, said heteroaryl optionally being mono- or polysubstituted, advantageously it is optionally substituted with 1 to 4 groups independently selected from:
a halogen atom,
an oxo group,
a hydroxyl,
a C₁-C₆ alkoxyl,
a C₁-C₆ alkyl, wherein 1 or several hydrogen atoms is (are) optionally replaced with a fluorine atom, and
a C₆-C₁₀ aryl optionally substituted with a halogen atom and/or a C₁-C₆ alkyl, preferably a fluorophenyl (in particular 4-fluorophenyl) or a terazole substituted with a C₁-C₆ alkyl such as a methyl;
when L represents —C(O)NH— or —NHC(O)—, W is selected from:
a C₆-C₁₀ aryl optionally mono- or polysubstituted, advantageously it is optionally substituted with a halogen atom, a hydroxyl, a C₁-C₆ alkyl, or a C₁-C₆ alkoxyl, preferably a fluorine atom, a hydroxyl, a methoxy or a trifluoromethyl, even more preferably a hydroxyl,
a 5-10 membered heteroaryl comprising from 1 to 2 heteroatoms independently selected from N, O and S, such as a furan, pyrrole, imidazole, pyrazole, thiophene, pyridine, pyrimidine, benzofuran, benzodioxole, indole, benzimidazole, said heteroaryl optionally being mono- or polysubstituted, advantageously it is optionally substituted with 1 to 4 substituents independently selected from:
a halogen atom,
a hydroxyl,
an oxo group,
a C₁-C₆ alkoxyl,
a C₁-C₆ alkyl, wherein 1 or several hydrogen atoms is (are) optionally replaced with a fluorine atom, and
a C₆-C₁₀ aryl optionally substituted with a halogen atom and/or a C₁-C₆ alkyl, preferably a fluorophenyl (in particular 4-fluorophenyl); and
a C₃-C₆ cycloakyl optionally substituted with a C(O)NHR or NHC(O)R group wherein R represents a C₆-C₁₀ aryl optionally substituted with a halogen atom and/or a C₁-C₆ alkyl, in particular a phenyl optionally substituted with a halogen atom and/or a C₁-C₆ alkyl, preferably a phenyl optionally substituted with a halogen atom, in particular it is substituted cyclopropyl with the formula 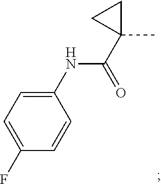
Cy represents a phenyl or a 5-10 membered heteroaryl containing from 1 to 3 heteroatoms independently selected from N, O and S, said heteroaryl optionally being substituted with an oxo group,
or a pharmaceutically acceptable salt thereof or a mixture thereof, for use in the prevention and/or treatment of viral infections.

2. The compound for use according to claim 1, of formula (Ia): 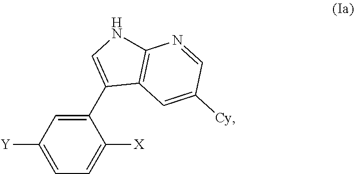
wherein X, Y and Cy are as defined in claim 1.

3. The compound for use according to claim 1, characterized in that Cy represents a phenyl, thiophene, furan, pyridine or pyrimidine group, in particular a phenyl or a thiophene.

4. The compound for use according to claim 1, characterized in that X represents a halogen, in particular fluorine.

5. The compound for use according to claim 1, characterized in that Y represents L-(CH₂)ₚ-W, L representing CH₂NH, p being an integer from 0 to 2, and W being selected from:
a phenyl optionally substituted with 1 or 2 groups independently selected from a bromine atom, a fluorine atom, a hydroxyl, methoxy, a trifluoromethyl, or triazole group, and
a heteroaryl selected from a furan, pyrrole, imidazole, pyrazole, thiophene, pyridine, pyrimidine, benzofuran, benzodioxole, indole and benzimidazole, said heteroaryl being optionally substituted with 1 to 4 groups independently selected from a halogen atom (in particular fluorine or bromine), a hydroxyl, an oxo group, a methoxy, a methyl, a trifluoromethyl, a phenyl and a fluorophenyl.

6. The compound for use according to claim 5, characterized in that W is an imidazole.

7. The compound for use according to claim 1, characterized in that Y represents NHC(O)—W, W being selected from:
2(1H)-pyridinone optionally substituted with 1 to 2 substituents independently selected from C₆-C₁₀ aryl optionally substituted with a halogen atom, preferably a fluorophenyl, in particular 4-fluorophenyl, and a methyl, and
a C₃-C₆ cycloakyl optionally substituted with a —C(O)NHR or —NHC(O)R group wherein R represents a C₆-C₁₀ aryl, in particular a phenyl, optionally substituted with a halogen atom, in particular substituted cyclopropyl of formula

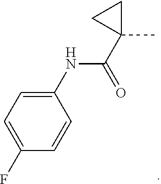

8. A compound for use according to claim 1, selected from: 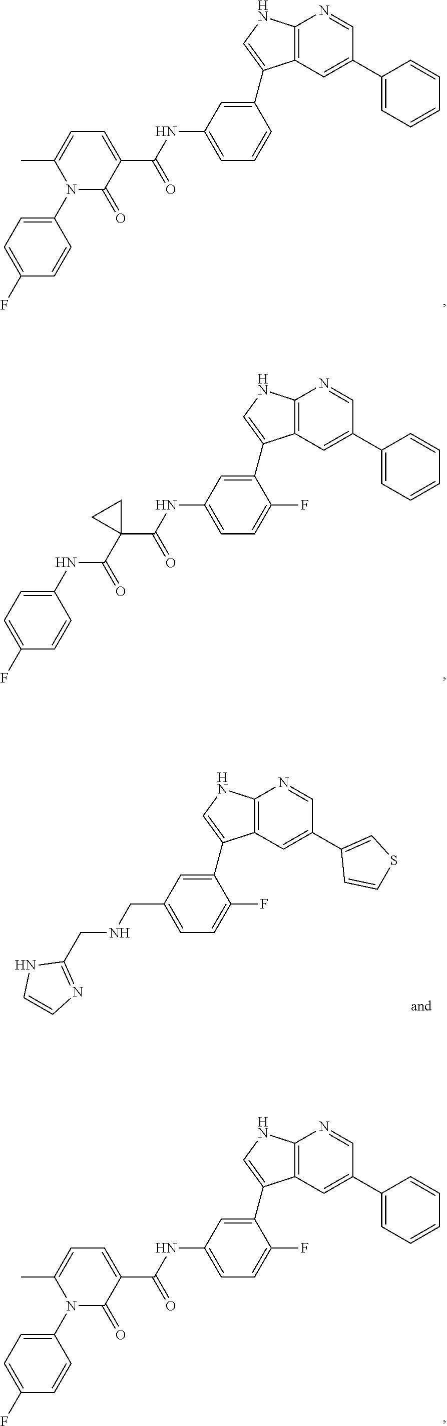 [SMILES: Cc1ccc(C(=O)Nc2cccc(-c3c[nH]c4ncc(-c5ccccc5)cc34)c2)c(=O)n1-c1ccc(F)cc1 ; O=C(Nc1ccc(F)cc1)C1(C(=O)Nc2ccc(F)c(-c3c[nH]c4ncc(-c5ccccc5)cc34)c2)CC1 ; Fc1ccc(CNCc2ncc[nH]2)cc1-c1c[nH]c2ncc(-c3ccsc3)cc12 ; Cc1ccc(C(=O)Nc2ccc(F)c(-c3c[nH]c4ncc(-c5ccccc5)cc34)c2)c(=O)n1-c1ccc(F)cc1]
preferably

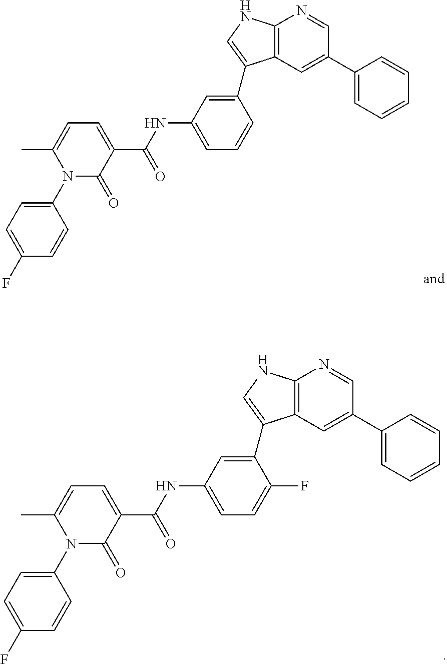 [SMILES: Cc1ccc(C(=O)Nc2cccc(-c3c[nH]c4ncc(-c5ccccc5)cc34)c2)c(=O)n1-c1ccc(F)cc1 ; Cc1ccc(C(=O)Nc2ccc(F)c(-c3c[nH]c4ncc(-c5ccccc5)cc34)c2)c(=O)n1-c1ccc(F)cc1]

9. A pharmaceutical composition comprising a compound according to claim 1 or a pharmaceutically acceptable salt thereof as an active ingredient, and a pharmaceutically acceptable excipient for use in the prevention and/or treatment of viral infections.

10. The pharmaceutical composition for use according to claim 9, further comprising an additional therapeutic agent, chosen from an antiviral agent, an anti-inflammatory agent, an immunomodulatory or immunosuppressive agent, an agent for the treatment of immunodeficiency disorders and an agent for pain treatment.

11. A compound for use according to claim 1, wherein the viral infections are due to Flaviviruses, such as dengue virus, Zika virus, West Nile virus, Kunjin virus, Alphaviruses, such as Chikungunya virus, Mayaro virus, Semliki Forest virus, Sindbis virus, Eastern equine encephalitis virus, Western equine encephalitis virus, Venezuelan equine encephalitis virus, Filoviruses such as Ebola virus, Marburg fever virus, arenaviruses such as Lassa fever virus, Junin virus, Amapari virus, picornaviruses such as vesicular stomatitis virus, paramyxoviruses such as influenza virus, or coronaviruses such as SARS-COV2.

12. A composition for use according to claim 9, wherein the viral infections are due to Flaviviruses, such as dengue virus, Zika virus, West Nile virus, Kunjin virus, Alphaviruses, such as Chikungunya virus, Mayaro virus, Semliki Forest virus, Sindbis virus, Eastern equine encephalitis virus, Western equine encephalitis virus, Venezuelan equine encephalitis virus, Filoviruses such as Ebola virus, Marburg fever virus, arenaviruses such as Lassa fever virus, Junin virus, Amapari virus, picornaviruses such as vesicular stomatitis virus, paramyxoviruses such as influenza virus, or coronaviruses such as SARS-COV2.
